(19) United States
(12) Patent Application Publication
(10) Pub. No.: US 2024/0404660 A1
(43) Pub. Date: Dec. 5, 2024

(54) MYOPIA DIAGNOSTIC AND PREVENTATIVE MODALITIES

(71) Applicant: APPLE INC., Cupertino, CA (US)

(72) Inventors: Adam W. Ward, San Diego, CA (US); Henning Zimmer, Zürich (CH); Jonathan T. Natale, Arcadia, CA (US)

(73) Assignee: APPLE INC., Cupertino, CA (US)

(21) Appl. No.: 18/632,961

(22) Filed: Apr. 11, 2024

Related U.S. Application Data
(60) Provisional application No. 63/470,435, filed on Jun. 1, 2023.

(51) Int. Cl. G16H 10/60

(52) U.S. Cl. CPC G16H 10/60

(57) ABSTRACT

A process may include detecting, by a user device at a first time, a distance between a display of the user device and a user. The process may include determining contextual information corresponding to a state of the user device at the first time. The process also may include determining an eye health event associated with the first time based at least in part on the distance and the contextual information. The process also may include performing an action relating to the eye health event based at least in part on the eye health event and other eye health events that occurred during other times, following the first time. The process also may then include generating an entry in a health datastore in response to determining the eye health event. The entry may include information about the eye health event and the contextual information.

### CROSS-REFERENCES TO OTHER APPLICATIONS

This application claims priority to U.S. Provisional Application No. 63/470,435, for “MYOPIA DIAGNOSTIC AND PREVENTATIVE MODALITIES” filed on Jun. 1, 2023, which is herein incorporated by reference in its entirety for all purposes.

### BACKGROUND

This application generally related to the eye health of device users. Specifically, this application related to distance-related viewing issues of device users.

### BRIEF SUMMARY

In an embodiment, a process may include detecting, by a user device at a first time, a distance between a display of the user device and a user associated with the user device. The process may include determining, by the user device, contextual information corresponding to a state of the user device at the first time. The process also may include determining, by the user device, an eye health event associated with the first time based at least in part on the distance and the contextual information. The process also may include performing, by the user device, an action relating to the eye health event based at least in part on the eye health event and other eye health events that occurred during other times, following the first time. The process also may then include generating, by the user device, an entry in a health datastore in response to determining the eye health event. The entry may include information about the eye health event and the contextual information.

In some embodiments, the process may include assigning, by the user device, a point to the eye health event. The process may then include determining, by the user device, that a point total of eye health including the point has reached a predetermined threshold. In response to determining that points total has reached the predetermined threshold, the process may include performing the action. In some embodiments, assigning the point is based in part on at least one of the eye health event or the contextual information. In some embodiments, the process may include augmenting a health record associated with the user to include the entry. The health record may be stored on the user device.

In some embodiments, the process may include generating a recommendation relating to eye health of the user, based at least in part on the entry in the health datastore. The process may also include accessing health data associated with the user, accessed from a remote server, and generating the recommendation relating to the eye health of the user based at least in part on the entry in the health datastore and the health data. The process may include performing the action is based at least in part on the entry in the health datastore.

In some embodiments, detecting the distance may also include gathering, by the user device, image data including a face of the user. The process may also include identifying, by the user device, the face of the user. The process may then include labelling, by the user device, the entry in the health datastore such that the eye health event is identified with the user. In some embodiments, the action relating to the eye health event may include displaying a notification on the user device, occluding a portion of the display of the user device, turning off the display of the user device, activating one or more haptic devices included in the user device, and/or transmitting a notification to a computing device.

In an embodiment a computing device may include a camera, a display, a display, one or more processors and a memory. The memory may include instructions that, when executed by the one or more processors, cause the computing device to perform certain operations. The computing device may detect, at a first time, a distance between the display and a user associated with the computing device. The computing device may then determine contextual information corresponding to a contextual of the computing device at the first time. The computing device may determine an eye health event associated with the first time based at least in part on the distance and the contextual information. The computing device may also perform an action relating to the eye health event based at least in part on the eye health event and other eye health events that occurred during other times following the first time. In response to determining the eye health event, the computing device may generate an entry in a health datastore. The entry may include information about the eye health event and the contextual information.

In some embodiments, the eye health event may include a myopic event related to a viewing distance. In some embodiments, the contextual information may include screen data, user interface data, a time, a duration, ambient light, a color of a portion of the display, a brightness of the display, an orientation of the user device, an orientation of the user, and/or application data associated with one or more applications running on the user device.

In some embodiments, the computing device may generate one or more ergonomic recommendations based at least in part on an orientation of computing device and an orientation of the user. The computing device may detect the distance occurs at regular intervals. The health datastore may be stored on the user device and may include health information collected by at least one of the user device or an accessory device.

In an embodiment, one or more non-transitory computer-readable media may include computer-executable instructions that, when executed by one or more processors of an electronic device, cause the electronic device to perform operations. The operations may include detecting, by a electronic device at a first time, a distance between a display of the electronic device and a user associated with the electronic device. The operations may include determining, by the electronic device, contextual information corresponding to a state of the electronic device at the first time. The operations also may include determining, by the electronic device, an eye health event associated with the first time based at least in part on the distance and the contextual information. The operations also may include performing, by the electronic device, an action relating to the eye health event based at least in part on the eye health event and other eye health events that occurred during other times, following the first time. The operations also may then include generating, by the electronic device, an entry in a health datastore in response to determining the eye health event. The entry may include information about the eye health event and the contextual information.

In some embodiments, the operations may include providing, by the electronic device, access to the eye health event in the health datastore to a computing device. Access to data related to the eye health event is provided via at least one of a publish/subscribe policy or an application programming interface. Detecting the distance may also include gathering, by the electronic device, image data including a face of the user. Detecting the distance may also include identifying, by the electronic device, the face of the user. Detecting the distance may then include labelling, by the electronic device, the entry in the health datastore such that the event is identified with the user.

In some embodiments, the action relating to the eye health event may include at least one of displaying a notification on the user device, occluding a portion of the display of the user device, turning off the display of the user device, activating one or more haptic devices included in the user device, and/or transmitting a notification to a computing device.

### BRIEF DESCRIPTION OF THE DRAWINGS

FIG. 1 illustrates a block diagram and a flowchart showing an example process for detecting an eye health event, according to at least one example.

FIG. 2 illustrates a simplified diagram of a user device and a user, according to at least one embodiment.

FIG. 3A illustrates a simplified diagram of orientations of a user device in relation to gravity, according to at least one embodiment.

FIG. 3B illustrates the simplified diagram of an orientation the user device with respect to a user, according to at least one embodiment.

FIG. 4 illustrates a block diagram of a system for determining an eye-health event, according to at least one embodiment.

FIG. 5 illustrates a process 500 for detecting an eye health event, according to at least one embodiment.

FIG. 6 illustrates a flowchart of a process for generating an entry in a health datastore and sharing associated health information, according to at least one embodiment.

FIG. 7 illustrates an example architecture or environment configured to implement techniques relating to detecting eye health events, according to at least one example.

### DETAILED DESCRIPTION

Adults and children alike are spending more and more time on user devices such as mobile phones, tablets, laptops, etc. Despite the warning of eye-health professionals worldwide, a user of these devices may often hold the display too close. For example, frequent viewing of displays at distances of less than 30 cm may lead to myopia or other eye health problems. Even without necessarily leading to myopia, repeated close viewing of displays is well-known to create eye strain and a loss in general productivity.

Sometimes, a user may view a device too closely due to poor habits or other reasons. The user may not have myopia, but after repeated viewing of displays too closely, the user may nevertheless become shortsighted. At other times, the user may already be diagnosed with myopia. Due to a change in eye condition, such as a change in prescription, the user may begin to view the user device closer than they previously did. In either case, there may be a need to detect an eye health event related to close-viewing of the display. For the non-myopia user, a corrective action may be performed, such as occluding the display for a time period in order to relieve eye strain or prevent eye damage. For the myopia user, the corrective action may be performed as well as some other action such as prompting the user to make an optometrist's appointment.

In a specific embodiment, a user device may use one or more sensors to gather image data and detect a distance between the user device and a user. The user device may also determine information relating to a state of the user device at the time the distance was detected. The image data and the information relating to the state of the user device may then be processed on-device to determine if an eye health event occurred. For example, the distance may be compared to a distance known to increase a risk myopia. If the detected distance is less than the known distance, an action may be performed by the user device. Furthermore, an entry may be made in a health datastore of the user device corresponding to the eye health event. The entry may include health-related information and/or other context-specific information associated with the eye health event (e.g., conditions present on the user device and surrounding the user device when the event was registered). The entry, and subsequent entries, may be later used to generate eye health recommendations for the user.

By detecting eye health events such as myopic events, the eye health of the user may be improved. In some embodiments, if an eye health event is detected, an action may be performed by the device. The action may be to display a notification, alter content being displayed at the time of the eye health event, occluding the display, or other such actions. For example, if the user device is displaying text when an eye health event occurs, the user device (or software included therein) may continuously increase a size of the text until the distance between the user device and user is increased beyond the certain threshold. In another example, the user device may move or vibrate until the user device is moved beyond the certain threshold. Thus, the user device may at least partially assist in improving and/or protecting the eye health of the user.

For example, the user may be alerted to the need for a new prescription or other medical need based on a history of eye health events. In some embodiments, a suggested new prescription may be generated and transmitted to a third party automatically. In other embodiments, a third party may be notified of an occurrence or occurrences of eye health events. A parent may be notified of their child's myopia-inducing behavior, for example. Examples of the present disclosure are directed towards detecting eye health events and some of the potential actions that may address the eye health events and/or the eye health of a user.

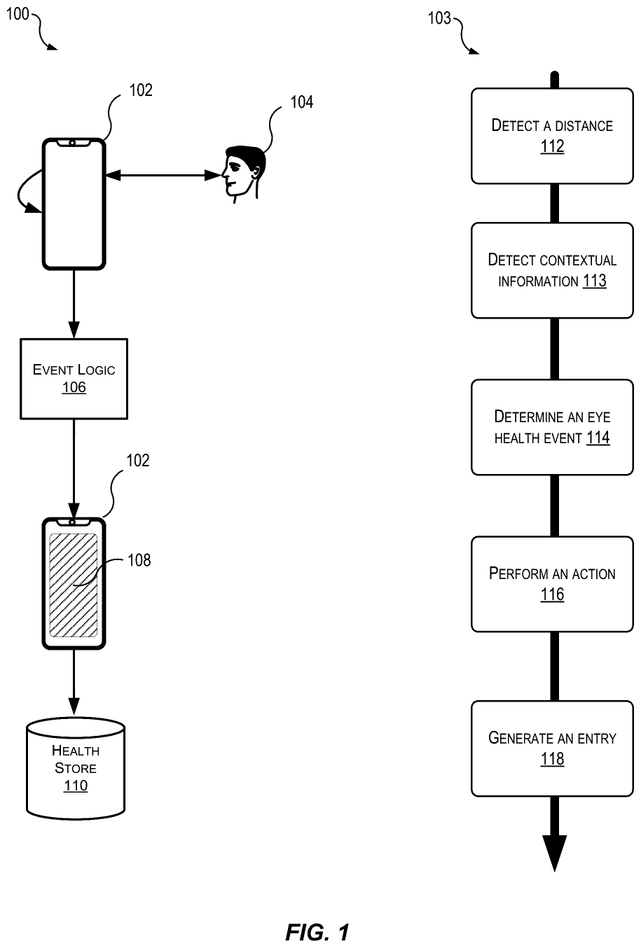

FIG. 1 illustrates a block diagram 100 and a flowchart showing an example 103 process for detecting an eye health event, according to at least one example. The diagram 100 may include a user device 102 and a user 104. The user device 102 is illustrated as a handheld portable user device such as a smartphone. As described herein, an example user device 102 may be any suitable user device such as a smartphone, tablet, media player, laptop, wearable device, and the like. In some examples, the user device 102 may include one or more applications, which may include custom-built algorithms and other logic, to enable performance of at least some of the techniques described herein. The user device 102 may also include storage media for storing computer-executable instructions (e.g., that make up the application) and other data such as described herein. The user devices 102 may be operated by one or more users such as the user 104.

FIGS. 1, 5, and 6 illustrate example flow diagrams showing processes 100, 500, and 600, according to at least a few examples. These processes, and any other processes described herein, are illustrated as logical flow diagrams, each operation of which represents a sequence of operations that can be implemented in hardware, computer instructions, or a combination thereof. In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer-readable storage medium, for example, in the form of a computer program including a plurality of instructions executable by one or more processors.

The user device 104 may be configured to capture image data via one or more image sensors and/or orientation data via one or more sensors. The user device 104 may include an event logic module 106. The event logic module 106 may include one or more rules and/or policies. The event logic module 106 may include a set of computer-readable instructions that cause the user device 104 to process data collected by the user device 104. For example, the event logic module 106 may then apply the one or more rules and/or policies to the data collected by the user device 104. When applied to the data collected by the user device 102, the event logic 106 can determine if an event is an eye health event, e.g., an event indicative of behavior of the user 104 that could negatively impact eye health.

The user device 102 may also include a health data store 110. The health data store 110 may include a logical partition of computer memory configured to store sensitive health data. In some embodiments, the health data store 110 may be implemented by a hardware component. The health data store 110 may be accessible only by a specific user (such as the user 104) of the user device 102. The specific user may permit data from the health data store 110 to be transmitted to an offline database. The specific user may permit data from the health data store 110 to be transmitted to one or more applications or third parties.

The process 103 may be performed by an application or applications running on the user device 102. Any actions performed by the user device 102 may be performed by and/or under the control of the application. For example, any or all of the process 103 may be performed by an operating system. The process 103 may begin at block 112 where the user device 102 detects a distance between the user device 102 and the user 104. The distance may be detected using a suitable sensor or package of sensors. For example, an infrared projector and an image sensor included in the user device 102 such as an infrared (IR) camera, or other suitable image sensor, may be used to detect a face of the user 104, a pose of the face (e.g., the face is looking at the camera), a distance between the face and the sensor, and/or make a binary decision if the face of user is closer than the predetermined threshold. The IR camera may detect the distance at regular intervals (e.g., every 20 seconds, 60 seconds, 80 seconds, etc.). In some embodiments, the user device 102 may only detect the distance while the user device 102 is in use. For example, the user device 102 may detect the distance at regular intervals while the display of the user device 102 is active, in response to some number of user inputs within the regular interval, or any other process or combination of process.

In detecting the distance, the user device 102 may utilize a distance model. The distance model may include functionality to determine if a face of the user 104 is within view. The distance model may determine that some or all of the face of the user 104 is in view and then determine a distance from the user device 102 to the face of the user. In some embodiments, the distance model may determine if eyes of the user 104 are gazing at the user device 102 and detect the distance in response to the detected gaze. If, by contrast the face of the user 104 is not in view and/or no gaze is detected, the user device 102 may not detect the distance. By only detecting distance when the gaze is with the user device 102 is detected, false positives may be reduced. For example, if the user is holding the mobile device 102 by their side and a distance is taken, the distance may not be relevant to eye health as no gaze is being made with the user device 102.

At block 113, the user device 102 may also determine contextual information associated with the user device 102 and/or the user 104 at the same time as the distance is determined. The contextual information may include information indicating a state of the user device 102. The contextual information may include information about applications running on the user device 104, user interface information, metadata such as a font size and screen brightness, one or more colors being displayed by the user device 104, and other such information. Because the contextual information may be determined at the same time as the distance, the contextual information may be linked to the distance as a single data entry or as correlated data entries.

At block 114, the user device 102 may process the distance and/or the contextual information to identify an eye-health event via the event logic module 106. The event logic module 106 may compare the distance to a preset parameter. The preset parameter may be a scientifically accepted distance, below which presents an eye health risk (e.g., 20 centimeters, 30 centimeters, etc.). If the distance is at or below the preset parameter, the user device 102 may identify an eye-health event relating to the distance and the contextual information. For example, the contextual information may indicate that the user 104 was interacting with a reading application at a first time. The distance at the first time may be 15 cm. Thus, the event logic module may determine that the first time corresponds to an eye-health event-namely that the user 104 was holding the user device 102 too closely while reading.

The event logic module 106 may also include a point total relating to eye health events. For example, every time the user device 102 determines an event is an eye health event via the event logic module 106, a point may be added to the point total. In some embodiments, the point total may be a running total, where the point total is persistent throughout a life of the user device 102. In other embodiments, the point total may reset to zero upon reaching the point total.

In other embodiments, the point total may reset to zero after a period of non-use. For example, the user device 102 may determine a series of eye-health events in a first time period (e.g., one hour). The user device 102 may then not be used for a second time period. The user device 102 may recognize the second time period as a period of non-use (e.g., as a result of no display activity registered). The user device 102 may then reset the point total to zero.

In yet another embodiment, the user device 102 may reset the point total in response to an eye health break. The eye health break may be detected using one or more sensors included in the user device 102. For example, the user device 102 may detect movement using one or more gyroscopes and a change in ambient light. The user device 102 may determine that the movement and change in ambient light represent a user of the user device 102 going outdoors and register an eye health break. In some embodiments, the point total may not reset to zero, but instead gradually reduce over time until finally reaching zero. One familiar in the art would recognize many different possibilities and configurations.

In certain embodiments, when the point total reaches a predetermined threshold (e.g., 2, 4, 8, etc. points), the user device 102 may perform an action. For example, the user device 102 may generate an occluded display 108. The occluded display 108 may include a solid color (e.g., black), display a pattern, cause the display to be blurry or out of focus, turn the display off, or any other suitable obfuscation of the display. The occluded display 108 may be persist for a preset time frame, such as twenty seconds. In some embodiments, the preset time frame may be based on the point total. For example, if the point total is four, the occluded display 108 may persist for ten seconds. If the point total were eight, the occluded display 108 may persist for twenty seconds. Other time frames are also possible.

Additionally or alternatively, the user device 102 may perform other actions. In some embodiments, the action may include a vibration or motion generated by one or more motors and/or haptic devices included in the user device 102. The vibration or motion may persist until the user device 102 detects that the distance has increased and/or the display has been turned off by the user 104. In some embodiments, the action may include transmitting a signal that causes a notification on a second device. For example, the user 104 may be a child and the user device 102 their tablet. In response to determining that the point total for the user device 102 is at or above the predetermined threshold, the user device 102 may transmit a notification to a device associated with the child's parent.

Any of the actions described may be performed, alone or in combination with any other actions. Other actions may include, turning the user device 102 off, displaying a notification on the user device 102, altering the display based on the distance, or any other suitable action.

At block 118, the user device 102 may generate an entry in the health data store 110. The entry may include the time the distance was determined, the distance, and/or the contextual information. The point total may also be stored in the health data store 110.

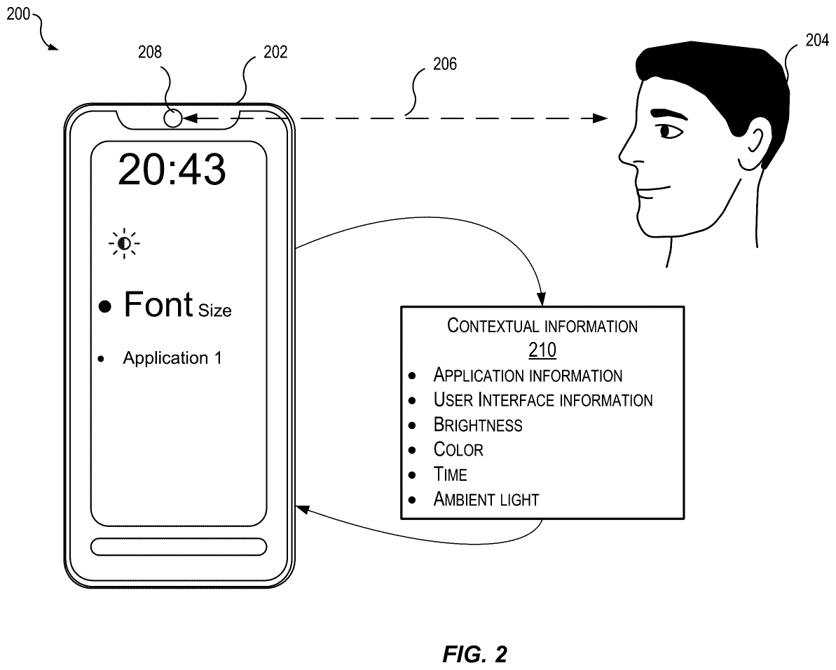

FIG. 2 illustrates a simplified diagram 200 of a user device 202 and a user 204, according to at least one embodiment. The user device 202 may be similar to the user device 102 in FIG. 1 and therefore include similar components and/or functionality. The user device 202 is illustrated displaying a time and various information about the user device 202 such as brightness and a font size. This illustration is merely a representative example, the user device 202 may be displaying some or none of the illustrated information. The user device 202 may display any suitable content of any suitable application. The applications may include a game, a video player, an image viewer, a text reader and other such applications.

The user device 202 may determine a distance 206 between a user 204 and a display of the user device 202 via an image sensor 208. The image sensor 208 may include one or more sensors such as a camera, an IR camera, a Lidar array, or any other suitable sensor. In some embodiments, the image sensor 208 may include a combination of sensors, such an IR camera and a Lidar array. The image sensor 208 may be a passive sensor, collecting image data that enters the image sensor 208 through an aperture. The image sensor 208 may also be an active sensor (e.g., a Lidar array), transmitting a signal and collecting data from a reflection of the signal. In some examples, the image sensor 208 may also enable certain functionalities of the user device, e.g., conducting face scans to unlock the user device 202.

In some embodiments, the user device 202 may determine the distance to the user 204 based at least in part on the application being used at the time. For example, a first application might be a text reader, displaying text for the user 204. The user device 202 may apply first settings associated with the first application that cause the mobile device to detect the distance 206. By contrast a second application may be associated with second settings. The second setting may cause the user device 202 to not perform the distance detection. In some embodiments, the distance detecting techniques described herein are enabled or disabled by a user input. The user input may be made on the user device 202 or on a separate device, associated with the user device 202. In some embodiments, the distance detection techniques may be enabled by default setting in an operating system of the user device 202.

The user device 202 may include a distance model. The distance model may include functionality to determine if a face of the user 204 is within view. The distance model may determine that some or all of the face of the user 204 is in view and then determine the distance 206 from the user device 202 to the face of the user. In some embodiments, the distance model may determine if the user 204 is gazing at the user device 202 and detect the distance in response to determining the gaze is fixed on the user device 202. If, by contrast the face of the user 204 is not in view and/or no gaze is detected, the user device 202 may not detect the distance. By only detecting distance when the gaze is fixed on the user device 202, false positives may be reduced. For example, if the user is holding the mobile device 202 by their side and a distance is taken, the distance may not be relevant to eye health as no gaze is fixed on the user device 202. Thus, the distance 206 may only represent the distance between the display of the user device 202 and the face of the user 204.

The user device 202 may determine contextual information 210 at the time the distance 206 is detected. The contextual information 210 may include the time, a brightness of the display, user interface information (e.g., font size), application information, a color of the display and/or other information corresponding to a context of the use device at the first time. The contextual information 210 may also include information about the user and/or the environment around the user. The image sensor 208, or other suitable sensor(s) of the user device 202, may also detect information about the environment around the user 204. For instance, the image sensor 208 may detect ambient light from the environment. The ambient light may indicate whether the user 204 is indoors or outdoors, a lighting type and/or lighting level being employed by the user 204, and other such information. The ambient light may be included in the contextual information 210.

In another example, via the image sensor 208, the user device 202 may recognize a face whose gaze is fixed on the user device 202. The distance model may fail to identify the face as that of the user 204, however. The user device 202 may indicate that the face is unrecognized in the contextual information 210. Thus, if the distance 206 is determined to be an eye health event, a point may not be added to the point total as the eye health event may not be associated with the user.

In yet another example, the user device 202 may detect multiple faces gazing at the user device 202. The distance model may then identify one or more the faces as one or more respective users. The user device 202 may then detect a respective distance to each of the faces and associate each respective distance with the respective user. The user device 202 may then determine one or more eye health events associated with each respective user and add points to a respective point total accordingly. In other examples, the user device 202 may determine an average of the respective distances. If the average is below the certain threshold and thus determined to be an eye health event, the user device may add a point accordingly. In yet another example, the user device 202 may identify the smallest distance of the respective distances. The user device 202 may then determine that the smallest distance is associated with an eye health event and add a point to the point total accordingly.

The user device 202 may use all or some of the contextual information 210 to determine an appropriate action. For example, if one color is displayed for a series of eye health events, the action may include displaying a different color to reduce eye strain. In another example, if the contextual information 210 indicates that a brightness level of the display is too low, the action may include brightening the display. Similarly, the contextual information 210 may include information about a font size being displayed. The action may therefore include increasing the font size until the user device 202 detects that the distance 206 has increased beyond a certain point (e.g., 40 cm). The user device 202 may increase the font size on a system-level or on an application-specific level. Similarly, the user device 202 may apply any action on a system-level or an application-specific level.

In relation to FIG. 1, the user device 202 may detect the distance 206 and gather the contextual information at a regular interval. The regular interval may be a preset parameter or may be configurable by a user such as the user 204. In some embodiments, the interval may be determined based on one or more factors included in the contextual information 210. For instance, the contextual information 210 may include a battery level of the user device 202. The user device 202 may then alter the interval based on the battery level (e.g., increase the interval from 80 seconds to 120 seconds).

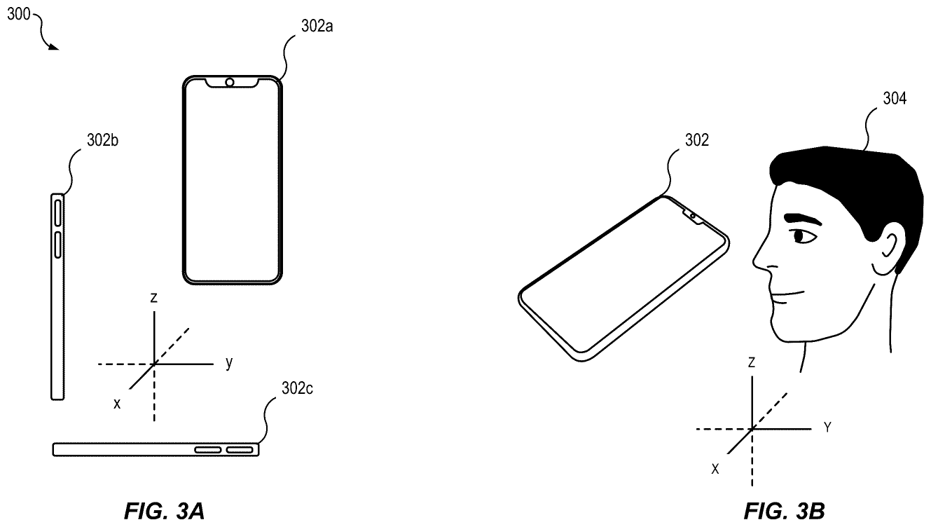

FIG. 3A illustrates a simplified diagram 300 of orientations 302a-c of a user device 302 in relation to gravity, according to at least one embodiment. FIG. 3B illustrates the simplified diagram of an orientation the user device 302 with respect to a user 304, according to at least one embodiment. The user device 302 may be similar to the user device 202 in FIG. 2 and have similar components and functionality. The coordinate axes in FIG. 3A may represent space in relation to gravity. The z-axis may correspond to “up and down,” with down being in-line with gravity. The x- and y-axes may therefore represent the other orthogonal axes.

For example, in the orientation 302a, the user device 302 may be oriented vertically along the z-axis, where the display of the user device is facing out along the x-axis. The user device 302 in orientation 302b may also be oriented vertically along the z-axis, but the display may be facing along the y-axis. In orientation 302c, the user device 302 may be oriented such that the face is pointed toward the z-axis. Using one or more gyroscopes, the user device 302 may be able to detect an orientation that is some combination of the orientations 302a-c.

In FIG. 3B, the coordinate axes may represent an orientation of the user 304. For example, the z-axis may extend through the top of user 304. The y-axis may extend out from the face of the user 304, and the x-axis may extend laterally across the face of the user 304. Using image data collected by the user device (e.g., via the image sensor 208 in FIG. 2), the user device 202 may determine an orientation of the user device 302 in relation to the user 204.

In an example orientation, the user 304 may be prone on a surface (e.g., floor, bed, couch, etc.) and the mobile device 302 may be lying flat on the surface. The user device 302 may therefore be in the orientation 302c with respect to gravity, with the display facing up along the z-axis. The user device 302 may therefore be oriented with the user 304 such that the display of the device is pointing towards the face of the user 304 (e.g., along the y-axis). If the top of the user device is oriented along the z-axis, the user device 302 may be in portrait mode. If the top of the user device 302 is oriented along the y-axis, the user device 302 may be in landscape mode. Other orientations would be readily apparent to one of ordinary skill in the art.

In any case, the orientation of the user device 302 in relation to gravity may be included in the contextual information 210 of FIG. 2. The orientation of the user device 302 in relation to the user 304 may also be included in the contextual information 210. Thus, actions performed by the user device 302 may be based at least in part on the orientation of the mobile device 302. For example, a notification may be generated on the user device 302, prompting the user 304 to change positions and/or move the user device 302 away. In other examples, the user device 302 may cause content displayed on the user device 302 to rotate or otherwise shift. The rotation of the content may correspond to the orientation 3A and/or 3B, rotating such that the user must move in a certain direct to clearly see the display.

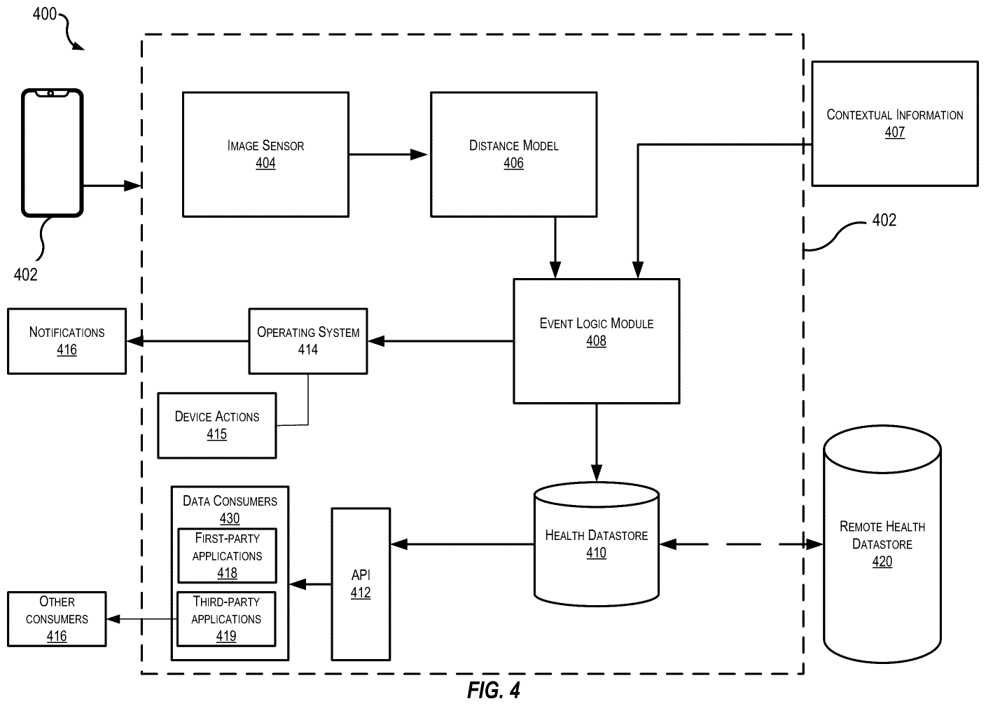

FIG. 4 illustrates a block diagram of a system 400 for determining an eye-health event, according to at least one embodiment. The system 400 may include a user device 402. The user device 402 may be similar to the user device 202 in FIG. 2. The user device 402 may also include other components not shown in FIG. 4, such as a computer memory, processors, gyroscopes and other sensors, etc. The user device 402 may be configured to perform any or all of the actions and processes described herein.

The user device 402 may include an image sensor 404, a distance model 406, an event logic module 408, and a health data store 410. The user device 402 may also include an application programming interface (API) 412. The API 412 may provide access to the health data store 410 to data consumers 430, according to privacy and access rules and policies. The data consumers 230 may include first-party applications 418, associated with the user device 402, such as a health app for managing health data of a user, calendar applications, map applications, or other applications that may utilize eye health information to make recommendations. The API 412 may also provide access to third-party applications 419. Third-party applications 419 may include e-commerce applications for generating recommendations based on health data, digital health records application, and other such applications. The third-party applications 419 may also transmit some of the health data accessed from the health data store 410 to other consumers 417. The other consumers 417 may include a web application (e.g., hosted by a doctor's office to access patient health data) or other such off-device data consumers. The image sensor 404 may include a camera, an IR camera, a Lidar array, or other such sensors. The image sensor 404 may contain any combination of these sensors. The image sensor 404 may be a passive sensor, collecting image data that enters the image sensor 404 through an aperture. The image sensor 404 may also be an active sensor (e.g., a Lidar array), transmitting a signal and collecting data from a reflection of the signal.

The image sensor 404 may provide image data to the distance model 406. The distance model 406 may utilize the image data to determine a distance between the user device 402 and a user. The distance model 406 may include functionality to determine if a face of a user is within view. The distance model 406 may determine that some or all of the face of the user is in view and then determine a distance from the user device 402 to the face of the user. In some embodiments, the distance model may determine if the user gazing at the user device 402 and detect the distance in response to the gaze being fixed on the user device 402. If, by contrast the face of the user 402 is not in view and/or no gaze is detected, the user device 402 may not detect the distance. By only detecting distance when the eye contact is fixed on the user device 402, false positives may be reduced. For example, if the user is holding the mobile device 402 by their side and a distance is taken, the distance may not be relevant to eye health as no is directed towards the user device 402.

The user device 402 may also determine contextual information 407. The contextual information 407 may be similar to the contextual information 210 in FIG. 2. Thus, the contextual information 407 may include information corresponding to a state of the user device and/or the user at the time the distance is determined. The contextual information 407 may include the time, a brightness of the display, user interface information (e.g., font size), application information, a color of the display and/or other information corresponding to a context of the use device at the first time. The contextual information may also include health-related information, accessed from the user device 402 or a remote device such as a wearable device (e.g., heart rate information, temperature information, movement information, etc.). The health-related information may also be accessed from the health datastore 410 and/or the remote health datastore 420 (e.g., information from the user's personal health record). The contextual information 407 may also include location information, information about whether the user is moving (e.g., sensor data), weather information, time information (e.g., date and time), state information from the device (e.g., whether alarm triggered), and any other information about the context in which the event is detected and which could be used to draw inferences and/or make recommendations about eye health. For example, it may be that the majority of the eye health events occur during the evening or when the user is lying down in bed. Based on this information, the techniques described herein can be used to not only inform the user of the eye health event (e.g., poor eye-health behavior) but may also make actionable recommendations (e.g., a recommendation for the user to avoid viewing certain content when in bed or during certain hours).

The contextual information 407 may also include information about the user and/or the environment around the user. The image sensor 404 may also detect information about the environment around the user. For instance, the image sensor 208 may detect ambient light from the environment. The ambient light may indicate whether the user is indoors or outdoors, a lighting type and/or lighting level being employed by the user, and other such information. The ambient light may be included in the contextual information 407.

After being processed by the distance model 406, the image data may be provided to the event logic module 408. The contextual information 407 may also be provided to the event logic module 408. The event logic module 408 may then further process the image data and/or the contextual information 407 to determine an eye health event. The event logic module 408 may compare the distance to a preset parameter. The preset parameter may be a scientifically accepted distance, below which presents an eye health risk (e.g., 20 centimeters, 30 centimeters, etc.). If the distance is at or below the preset parameter, the user device 402 may identify an eye-health event relating to the distance and the contextual information.

The event logic module 408 may also assign a point to the eye health event. The event logic module 408 may also include a point total, where each point relates to an eye health event. For example, every time the user device 402 determines an event is an eye health event via the event logic module 408, a point may be added to the point total. In some embodiments, the point total may be a running total, where the point total is persistent throughout a life of the user device 402.

The user device 402 may cause the point total to be stored in the health datastore 410 in connection with the contextual information eye event that caused the point to be counted. In response to the point total meeting or exceeding a predetermined threshold (e.g., four points), the user may perform an action, as is described above. The actions may include occluding the display of the user device 402, generating a notification on the user device 402 and/or a second device, generating an alert using one or more speakers and/or motors, or any other suitable action.

The user device 402 may also cause an entry including information about the eye health event and the contextual information in the health data store 410. The user device 402 may store an entry for every eye health event determined by the user device 402. Thus, the health datastore 410 may include a history of eye health events detected by the user device 402. In some embodiments the history of eye health events may be synchronized with other health data stored on the user device 402. The information from the health datastore 410 may be used by other applications to make predictions (e.g., identify trends in health data), populate dashboards (e.g., a dashboard that shows the frequency of eye-related events alone or together other health metrics such as minutes of screen time, sleep patterns, distance walked, etc.).

The event logic module 408 may transmit data associated with the entry to an operating system 414 of the user device 402. The operating system may then perform one or more device actions 415. The device actions 415 may include any of the actions described above. For example, the operating system 414 enlarge a font size of the system operating on the user device 402 in response to the entry. The operating system 414 may also cause the display to be occluded in response to the entry, as shown in FIG. 1. The operating system 414 may adjust other aspects of the display, such as a tone, brightness, and other such aspects.

The operating system may cause one or more notifications 416 to be generated. For example, the notifications 416 may include causing an email to be sent to a third-party in response to the entry. For example, if the user device 402 is a child's device, the notifications 416 may send an e-mail notification to a parent's email that an eye health event has occurred. In another embodiment, the notifications 416 may email a periodic report based on the history of eye health events.

The first-party applications 418 may access the history of eye health events from the health datastore 410 via the API 412. The first-party applications 418 may also access health data from the remote health datastore 420. The first-party applications 418 may analyze the history of eye health events and the health data to generate health information relevant to the user. For example, the first-party applications 418 may utilize the history of eye health events and the health event to recommend an eye exam due to a recent uptick in the occurrence of eye health events. The first-party applications 418 may then generate an email or other notification via the notifications 416. The email may be transmitted to an account associated with the user.

The user device 402 may also include other third-party applications 419. The third-party applications 419 may also access the entry and/or history of eye health events via the API 412. The third-party applications 419 may aggregate and utilize the entry and/or history of eye health events in any useful manner. In one example, one of the third-party applications 419 may be associated with an online retailer. This third-party application may access health data from the health datastore 410 via a pub/sub model, such that the third-party app receives health data (including the history of eye health events) on some regular publishing schedule of the health datastore 410. The third-party application may then use at least some of the health data to make any suitable recommendations for the user. For example, an ecommerce application may make one or more product recommendations (e.g., glasses, eye drops, etc.) to the user based on the context information and the eye health events.

In another example, the third-party applications 419 may include a health records management (HRM) application. The HRM application may access data from the health datastore 410 to create or update a heath record associated with the user. The HRM application may then transmit some or all of the health record to be transmitted to a remote server, according to privacy settings and policies as determined by a user. The HRM application (and/or the remote server) may also access health data from the remote health datastore 420 and update the health record accordingly.

In some embodiments, the third party applications 419 may be transmit health information based on one or more entries to a third party, such as an optometrist. For example, after some number of eye health events are recorded, the third-party application(s) may send a notification to the optometrist, schedule an appointment with the optometrist, or perform other such actions. In another embodiment, the third-party applications 419 may be used to update an ocular prescription of the user. In some examples, using the HRM application or other application such as a first-party application, the user device may share certain data types from the health datastore 410 (e.g., entries relating to eye-health events and corresponding contextual information) with other user devices (e.g., family members), with health care providers (e.g., optometrist), and others for any suitable duration. For example, this may include the user device establishing a sharing relationship with a device of the other user. In the example of the optometrist, the optometrist's device may use the API 412 to periodically retrieve the specific data type to which it is has been given access. The optometrist may use this information to generate a care plan for the user, which might include eye-health related suggestions.

The system 400 may also optionally include the remote health datastore 420. The remote health datastore 420 may be associated with a service provider of the user device 402. The user device 402 may send and receive health data associated with the user to and from the remote health datastore 420. The user device 402 may also allow the data consumers 430 to access health data from the remote health datastore 420 via the API 412, according to permissions set by the user. The user device 402 may also cause the entry and/or history of eye health events to be transmitted to the remote health datastore. In some embodiments, the entry may be transmitted from the event logic module 408 directly to the remote health datastore 420 (in other words, as an eye health event occurs). In other embodiments, the history of eye health events may be transmitted from the health data store 410 to the remote health datastore 420.

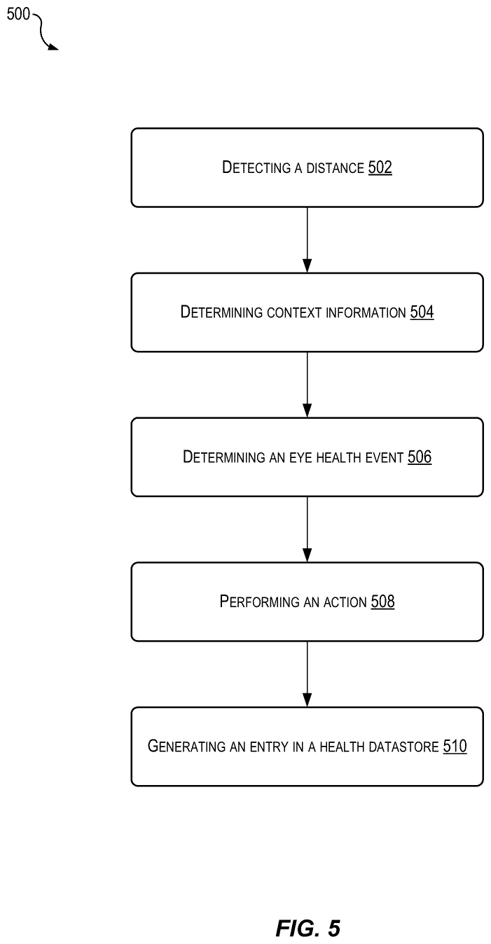

FIG. 5 illustrates a process 500 for detecting an eye health event, according to certain embodiments. The process 500 may be performed by any of the systems described herein, such as the user device 402 in FIG. 4. The process 500 may use a distance between a user device and contextual information about the device to determine an eye health event. The process 500 may also include performing actions by the user device to mitigate or remedy the eye health evet. At block 502, the process 500 includes detecting a distance between a user and a display of a user device at a first time. The user may be associated with the user device. The user device may be a laptop, tablet, smartphone, wearable device, or other such device.

Detecting the distance may include receiving image data from one or more image sensors. The image sensors may include a camera, an IR camera, a Lidar array, or other suitable image sensor. The image sensor may be a passive sensor, collecting image data that enters the image sensor through an aperture. The image sensor may also be an active sensor (e.g., a Lidar array), transmitting a signal and collecting data from a reflection of the signal.

The user device may detect the distance at regular intervals (e.g., every 20 seconds, 60 seconds, 80 seconds, etc.). In some embodiments, the user device may only detect the distance while the user device is in use. For example, the user device may detect the distance at regular intervals while the display of the user device is active, in response to some number of user inputs within the regular interval, or any other process or combination of process. The regular interval may be a preset parameter or may be configurable by a user such as the user.

In detecting the distance, the user device may utilize a distance model. The distance model may include functionality to determine if a face of the user is within view. The distance model may determine that some or all of the face of the user is in view and then determine a distance from the user device to the face of the user. In some embodiments, the distance model may determine if the user is making gazing at the user device and detect the distance in response to the gaze being fixed on the user device. If, by contrast the face of the user is not in view and/or no gaze is detected, the user device may not detect the distance. By only detecting distance when the gaze is fixed on the user device, false positives may be reduced. For example, if the user is holding the mobile device by their side and a distance is taken, the distance may not be relevant to eye health as no gaze is directed towards the user device.

At block 504, the process 500 includes determining contextual information corresponding to a state of the user device at the first time. The contextual information may include of screen data, user interface data, a time, a duration, ambient light, a color of a portion of the display, a brightness of the display, an orientation of the user device, an orientation of the user, or application data associated with one or more applications running on the user device.

The contextual information may also include information about the user and/or the environment around the user. For instance, the image sensor may detect ambient light from the environment. The ambient light may indicate whether the user is indoors or outdoors, a lighting type and/or lighting level being employed by the user, and other such information. The ambient light may be included in the contextual information.

At block 506, the process 500 includes determining an eye health event associated with the first time and based at least in part on the distance and the contextual information. The eye health event may include a myopic event related to a viewing distance. The eye health event may be determined by an event logic module such as the event logic module in FIG. 1. The event logic module may process the image data and/or the contextual information to determine an eye health event. The event logic module may compare the distance to a preset parameter. The preset parameter may be a scientifically accepted distance, below which presents an eye health risk (e.g., 20 centimeters, 30 centimeters, etc.). If the distance is at or below the preset parameter, the user device may identify an eye-health event relating to the distance and the contextual information.

The event logic module may also assign a point to the eye health event. The event logic module may also include a point total, where each point relates to an eye health event. For example, every time the user device determines an event is an eye health event via the event logic module, a point may be added to the point total. In some embodiments, the point total may be a running total, where the point total is persistent throughout a life of the user device.

At block 508, the process 500 includes performing an action relating to the eye health event. The action may be performed based at least in part on the eye health event and other eye health events that occurred following the first time. The action may include any or all of occluding the display of the user device, turning off the display of the user device, transmitting one or more notifications to a second device, displaying notifications, generating an alert using one or more motors, generating an auditory alert, or any other suitable action. In some embodiments, the action may be related to the point total reaching a predetermined threshold. In other embodiment, the action may be determined based at least in part on a history of eye health events.

At block 510, the process 500 includes generating an entry in a health datastore, responsive to determining the eye health event. The entry may include information about the eye health event and the contextual information. The health data store may be stored on the user device, such as the health datastore 410 in FIG. 4. The health data store may include health information collected by the user device and/or an accessory device (such as a wearable device). In some embodiments, the user device may augment a health record associated with the user to include the entry. For example, the health record may be the history of eye health events described in FIG. 4 or any other relevant health record.

In some embodiments, the process 500 may include generating a recommendation relating to eye health based at least in part on the entry. For example, the user device may generate a notification to make an appointment with an optometrist. The user device may additionally or alternatively recommend a new ocular prescription. The process 500 may also include accessing health data associated with the user from a remote server, such as the remote health datastore 420 in FIG. 4. In some embodiments, the user device may generate one or more ergonomic recommendations based on the orientation of the computing device and the user. The orientation of the user device and/or user may be determined by systems such is described in FIG. 3.

In some embodiments, the process 500 may include gathering image data including a face of the use via the one or more image sensors. The process 500 may also include identifying the face of the user, such as with the distance model described above. The process 500 may also include labelling, by the user device, the entry in the health datastore such that the event is identified with the user. In some embodiments, access to data related to the eye health event is provided by a publish/subscribe policy and/or an API, such as the API 412.

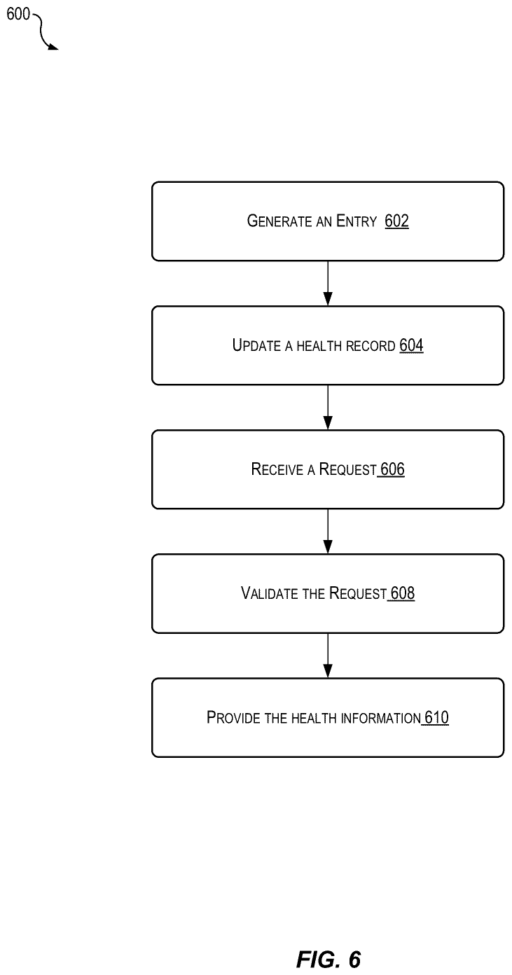

FIG. 6 illustrates a flowchart of a process 600 for generating an entry in a health datastore and sharing associated health information, according to at least one embodiment. The process 600 may be performed by any of the systems described herein, such as the user device 402 in FIG. 4. The process 600 may generate an entry in a health datastore and/or update a health record. Some or all of the health record may then be shared with one or more applications, according to privacy settings determined by a user.

At block 602, the process 500 includes generating, by a user device, an entry in a health datastore of the user device. The entry may be associated with an eye-health event and include a distance between the user device and the user. The distance may be determined by a camera or other sensor included on the user device. The entry may also include contextual information about the state of the user device at the time of the eye health event. At block 604, the process 600 may include updating a health record to include the entry. The health record may include a point associated with the eye health event (indicating that the eye health evet occurred), The distance associated with the eye health event, and or the contextual information. The health record may include past entries associated with previous eye health events and other health information relating to the eye health of the user (e.g., a prescription, eye diseases or maladies, etc.). The health record may also include other health information associated with the user, such as temperature, pulse, medical history, and other relevant health data. In some embodiments, a copy the health record may also be updated on a remote health data store, such as the remote health datastore 420 in FIG. 4.

At block 606, the process 600 includes receiving, by the user device, a request to access health information relating to the eye health of the user. The request may be received by an API (such as the API 412 in FIG. 4) from an application running on the user device, such as the first-party applications 418 and/or the third-party applications 419. In some embodiments, the request may be to update another health record associated with the user. In other embodiments, the request may be for some or all of the eye health information to be used to generate recommendations for the user. The recommendations may be for treatment of eye health issues, products, or a recommendation to make an optometrist's appointment.

At block 608, the process 600 includes validating the request based at least in part on one or more privacy policies. The one or more privacy policies may be stored on the user device 202 and accessed by the API. The API (or other software) may determine a sender associated with the request and any policies associated with the sender. The policies associated with each sender/application may indicate varying levels of permissiveness where not all health information is accessible to the sender. For example, one application may be associated with a research institution. An associated policy may indicate that only aggregated information may be provided to the research institution. Thus, the API may then determine that aggregated eye health information may be accessed by the research institution. In another example, the sender may be an online retailer. The associated policy may indicate that online retailer is permitted to access health information relating to the eye health of the user. The API may then determine that the heath information may be accessed. In yet another example, the sender may be a private account of an acquaintance of the user. The API may determine that the associated policy bars the access of any health information by the acquaintance.

At block 608, the process 600 includes providing, by the computing device, at least a portion of the health information relating to the eye health of the user. In providing the health information, the API may access the health record and or the entry(ies) in the datastore. In some embodiments, the API (or related software) may access information stored on a remote health datastore. The health information may be provided in response to the validation of the request. The provided health information may only include the permitted type and amount of health information (e.g., the aggregated health information described above). The health information may be provided via the API to an application such as the first- and third-party applications.

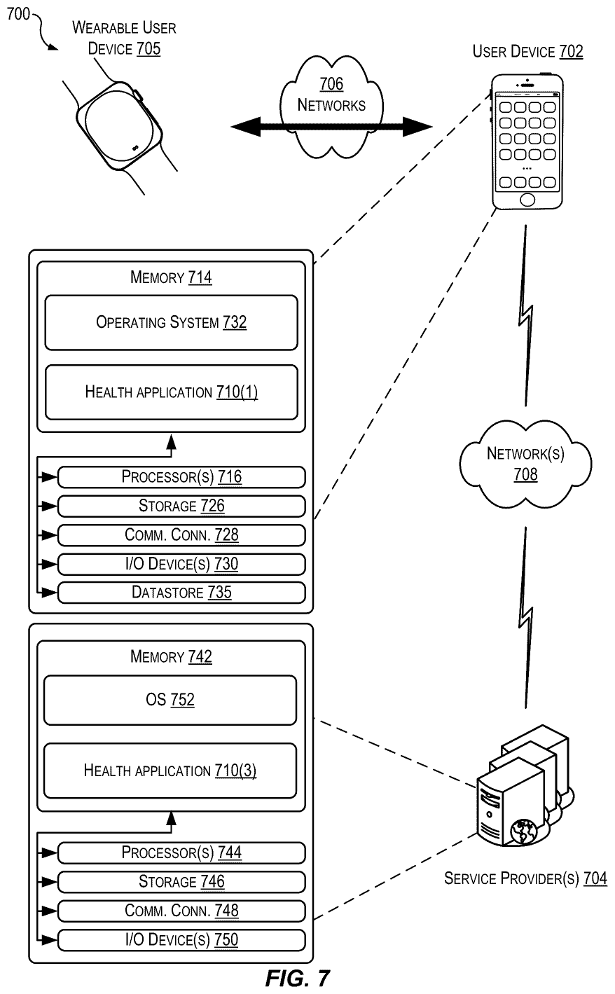

FIG. 7 illustrates an example architecture or environment 700 configured to implement techniques relating to detecting eye health events, according to at least one example. In some examples, the example architecture 700 may further be configured to enable a user device 702 (e.g., the user device 102), the service provider computers 704 (e.g., the service provider 704), and a wearable electronic device 705 (e.g., an example accessory deice) to share information. In some examples, the devices may be connected via one or more networks 708 and/or 706 (e.g., via Bluetooth, WiFi, the Internet, or the like). In the architecture 700, one or more users may utilize the user device 702 to manage, control, or otherwise utilize the wearable electronic device 705, via the one or more networks 706. Additionally, in some examples, the wearable electronic device 705, the service provider computers 704, and the user device 702 may be configured or otherwise built as a single device. For example, the wearable electronic device 705 and/or the user device 702 may be configured to implement the examples described herein as a single computing unit, exercising the examples described above and below without the need for the other devices described.

In some examples, the networks 706, 708 may include any one or a combination of many different types of networks, such as cable networks, the Internet, wireless networks, cellular networks, satellite networks, other private and/or public networks, or any combination thereof. While the illustrated example represents the user device 702 accessing the service provider computers 704 via the networks 708, the described techniques may equally apply in instances where the user device 702 interacts with the service provider computers 704 over a landline phone, via a kiosk, or in any other manner. It is also noted that the described techniques may apply in other client/server arrangements (e.g., set-top boxes, etc.), as well as in non-client/server arrangements (e.g., locally stored applications, peer to peer configurations, etc.).

The user device 702 may be any type of computing device such as, but not limited to, a mobile phone, a smartphone, a personal digital assistant (PDA), a laptop computer, a desktop computer, a thin-client device, a tablet computer, a wearable device, or the like. In some examples, the user device 702 may be in communication with the service provider computers 704 via the networks 708, 706, or via other network connections.

In one illustrative configuration, the user device 702 may include at least one memory 714 and one or more processing units (or processor(s)) 716. The processor(s) 716 may be implemented as appropriate in hardware, computer-executable instructions, firmware, or combinations thereof. Computer-executable instruction or firmware implementations of the processor(s) 716 may include computer-executable or machine-executable instructions written in any suitable programming language to perform the various functions described. The user device 702 may also include geo-location devices (e.g., a global positioning system (GPS) device or the like) for providing and/or recording geographic location information associated with the user device 702.

The memory 714 may store program instructions that are loadable and executable on the processor(s) 716, as well as data generated during the execution of these programs. Depending on the configuration and type of the user device 702, the memory 714 may be volatile (such as random access memory (RAM)) and/or non-volatile (such as read-only memory (ROM), flash memory, etc.). The user device 702 may also include additional removable storage and/or non-removable storage 726 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 714 may include multiple different types of memory, such as static random access memory (SRAM), dynamic random access memory (DRAM), or ROM. While the volatile memory described herein may be referred to as RAM, any volatile memory that would not maintain data stored therein once unplugged from a host and/or power would be appropriate.

The memory 714 and the additional storage 726, both removable and non-removable, are all examples of non-transitory computer-readable storage media. For example, non-transitory computer readable storage media may include volatile or non-volatile, removable or non-removable media implemented in any process or technology for storage of information such as computer-readable instructions, data structures, program modules, or other data. The memory 714 and the additional storage 726 are both examples of non-transitory computer storage media. Additional types of computer storage media that may be present in the user device 74 may include, but are not limited to, phase-change RAM (PRAM), SRAM, DRAM, RAM, ROM, Electrically Erasable Programmable Read-Only Memory (EEPROM), flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the user device 702. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media.

The user device 702 may also contain communications connection(s) 728 that allow the user device 702 to communicate with a data store, another computing device or server, user terminals, and/or other devices via the networks 708, 706. The user device 702 may also include I/O device(s) 730, such as a keyboard, a mouse, a pen, a voice input device, a touch input device, a display, an operating system 732 and/or one or more application programs or services for implementing the features disclosed herein including a health application 710(1). In some examples, the health application 710(1) may be configured to implement the features described herein such as those described with reference to the flowcharts. User device 702 may also include a datastore 735. The datastore 735 may be a separate memory partition within the memory 714 or may be an individual hardware component of the user device 702. The datastore 735 may be configured as a health datastore (e.g., the health datastore 410 in FIG. 4) as described above.

The service provider computers 704 may also be any type of computing device such as, but not limited to, a mobile phone, a smartphone, a PDA, a laptop computer, a desktop computer, a thin-client device, a tablet computer, a wearable device, a server computer, a virtual machine instance, etc. In some examples, the service provider computers 704 may be in communication with the user device 702 and/or the wearable user device 705 via the networks 708, 706, or via other network connections. The service provider computers 704 may also include the remote health datastore 420 in FIG. 4. Thus, the user device 702 may communicate with the remote health datastore via the service computers 704.

In one illustrative configuration, the service provider computers 704 may include at least one memory 742 and one or more processing units (or processor(s)) 744. The processor(s) 744 may be implemented as appropriate in hardware, computer-executable instructions, firmware, or combinations thereof. Computer-executable instruction or firmware implementations of the processor(s) 744 may include computer-executable or machine-executable instructions written in any suitable programming language to perform the various functions described.

The memory 742 may store program instructions that are loadable and executable on the processor(s) 744, as well as data generated during the execution of these programs. Depending on the configuration and type of service provider computer 704, the memory 742 may be volatile (such as RAM) and/or non-volatile (such as ROM, flash memory, etc.). The service provider computer 704 may also include additional removable storage and/or non-removable storage 746 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 742 may include multiple different types of memory, such as SRAM, DRAM, or ROM. While the volatile memory described herein may be referred to as RAM, any volatile memory that would not maintain data stored therein once unplugged from a host and/or power would be appropriate. The memory 742 and the additional storage 746, both removable and non-removable, are both additional examples of non-transitory computer-readable storage media.

The service provider computer 704 may also contain communications connection(s) 748 that allow the service provider computer 704 to communicate with a data store, another computing device or server, user terminals and/or other devices via the networks 708, 706. The service provider computer 704 may also include I/O device(s) 750, such as a keyboard, a mouse, a pen, a voice input device, a touch input device, a display, speakers, a printer, etc. The memory 742 may include an operating system 752 and/or one or more application programs or services for implementing the features disclosed herein including the health application 710(3). This version of the health application may be configured to perform similar operations as the health datastore 710(1). Thus, in some examples, the health application 710(3) may be configured to implement the features described herein such as those described with reference to the flowcharts.

The various examples further can be implemented in a wide variety of operating environments, which in some cases can include one or more user computers, computing devices or processing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system also can include a number of workstations running any of a variety of commercially available operating systems and other known applications for purposes such as development and database management. These devices also can include other electronic devices, such as dummy terminals, thin-clients, gaming systems, and other devices capable of communicating via a network.

Most examples utilize at least one network that would be familiar to those skilled in the art for supporting communications using any of a variety of commercially available protocols, such as TCP/IP, OSI, FTP, UPnP, NFS, CIFS, and AppleTalk. The network can be, for example, a local area network, a wide-area network, a virtual private network, the Internet, an intranet, an extranet, a public switched telephone network, an infrared network, a wireless network, and any combination thereof.

In examples utilizing a network server, the network server can run any of a variety of server or mid-tier applications, including HTTP servers, FTP servers, CGI servers, data servers, Java servers, and business application servers. The server(s) may also be capable of executing programs or scripts in response to requests from user devices, such as by executing one or more applications that may be implemented as one or more scripts or programs written in any programming language, such as Java®, C, C# or C++, or any scripting language, such as Perl, Python or TCL, as well as combinations thereof. The server(s) may also include database servers, including without limitation those commercially available from Oracle®, Microsoft® Sybase®, and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of examples, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as RAM or ROM, as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a non-transitory computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or browser. It should be appreciated that alternate examples may have numerous variations from that described above. For example, customized hardware might also be used and/or particular elements might be implemented in hardware, software (including portable software, such as applets) or both. Further, connection to other computing devices such as network input/output devices may be employed.

Non-transitory storage media and computer-readable media for containing code, or portions of code, can include any appropriate media known or used in the art, including storage media, such as, but not limited to, volatile and non-volatile, removable and non-removable media implemented in any method or technology for storage of information such as computer-readable instructions, data structures, program modules, or other data, including RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information and which can be accessed by a system device. Based at least in part on the disclosure and teachings provided herein, a person of ordinary skill in the art will appreciate other ways and/or methods to implement the various examples.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (e.g., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate examples of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Preferred examples of this disclosure are described herein, including the best mode known to the inventors for carrying out the disclosure. Variations of those preferred examples may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

The present disclosure recognizes that the use of such personal information data, in the present technology, can be used to the benefit of users. For example, the personal information data can be used to provide a family member or friend a view of health data updates. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed blocks for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the U.S., collection of or access to certain health data may be governed by federal and/or state laws, such as the Health Insurance Portability and Accountability Act (HIPAA); whereas health data in other countries may be subject to other regulations and policies and should be handled accordingly. Hence, different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services or other services relating to health record management, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health-related applications, data de-identification can be used to protect a user's privacy. De-identification may be facilitated, when appropriate, by removing specific identifiers (e.g., date of birth), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

What is claimed is:

1. A computer-implemented method, comprising:
detecting, by a user device at a first time, a distance between a display of the user device and a user associated with the user device;
determining, by the user device, contextual information corresponding to a state of the user device at the first time;
determining, by the user device, an eye health event associated with the first time based at least in part on the distance and the contextual information;
performing, by the user device, an action relating to the eye health event based at least in part on the eye health event and other eye health events that occurred during other times following the first time; and
responsive to determining the eye health event, generating, by the user device, an entry in a health datastore, the entry including information about the eye health event and the contextual information.

2. The computer-implemented method of claim 1, further comprising:
assigning, by the user device, a point to the eye health event;
determining, by the user device, that a point total of eye health events comprising the point has reached a predetermined threshold; and
in response to determining that points total has reached the predetermined threshold, performing the action.

3. The computer-implemented method of claim 2, wherein assigning the point is based in part on at least one of the eye health event or the contextual information.

4. The computer-implemented method of claim 1, further comprising:
augmenting, by the user device, a health record associated with the user to include the entry, the health record stored on the user device.

5. The computer-implemented method of claim 1, further comprising:
generating a recommendation relating to eye health of the user based at least in part on the entry in the health datastore.

6. The method of claim 5, further comprising:
accessing health data associated with the user, the health data accessed from a remote server; and
generating the recommendation relating to the eye health of the user based at least in part on the entry in the health datastore and the health data.

7. The computer-implemented method of claim 1, wherein performing the action is based at least in part on the entry in the health datastore.

8. The computer-implemented method of claim 1, wherein detecting the distance further comprises:
gathering, by the user device, image data including a face of the user;
identifying, by the user device, the face of the user; and
labelling, by the user device, the entry in the health datastore such that the eye health event is identified with the user.

9. The computer-implemented method of claim 1, wherein the action relating to the eye health event includes at least one of displaying a notification on the user device, occluding a portion of the display of the user device, turning off the display of the user device, activating one or more haptic devices included in the user device, or transmitting a notification to a computing device.

10. A computing device, comprising:
a camera;
a display;
one or more processors; and
a memory comprising instructions that, when executed by the one or more processors, cause the computing device to perform operations to:
detect, at a first time, a distance between the display and a user associated with the computing device;
determine contextual information corresponding to a contextual of the computing device at the first time;
determine an eye health event associated with the first time based at least in part on the distance and the contextual information;
perform an action relating to the eye health event based at least in part on the eye health event and other eye health events that occurred during other times following the first time; and
responsive to determining the eye health event, generate an entry in a health datastore, the entry including information about the eye health event and the contextual information.

11. The computing device of claim 10, wherein the eye health event comprises a myopic event related to a viewing distance.

12. The computing device of claim 10, wherein the contextual information comprises at least one of screen data, user interface data, a time, a duration, ambient light, a color of a portion of the display, a brightness of the display, an orientation of the user device, an orientation of the user, or application data associated with one or more applications running on the user device.

13. The computing device of claim 10, further comprising:
generating, by the computing device, one or more ergonomic recommendations based at least in part on an orientation of computing device and an orientation of the user.

14. The computing device of claim 10, wherein detecting the distance occurs at regular intervals.

15. The computing device of claim 10, wherein the health datastore is stored on the user device and includes health information collected by at least one of the user device or an accessory device.

16. One or more non-transitory computer-readable media comprising computer-executable instructions that, when executed by one or more processors of an electronic device, cause the one or more processors to perform operations comprising:
detecting, by the electronic device at a first time, a distance between a display of the electronic device and a user associated with the electronic device;
determining, by the electronic device, contextual information corresponding to a contextual of the electronic device at the first time;
determining, by the electronic device, an eye health event associated with the first time based at least in part on the distance and the contextual information;
performing, by the electronic device, an action relating to the eye health event based at least in part on the eye health event and other eye health events that occurred during other times following the first time; and
responsive to determining the eye health event, generating, by the electronic device, an entry in a health datastore, the entry including information about the eye health event and the contextual information.

17. The one or more non-transitory computer-readable media of claim 16, the operations further comprising:
providing, by the electronic device, access to the eye health event in the health datastore to a computing device.

18. The one or more non-transitory computer-readable media of claim 17, wherein access to data related to the eye health event is provided via at least one of a publish/subscribe policy or an application programming interface.

19. The one or more non-transitory computer-readable media of claim 16, wherein detecting the distance further comprises:
gathering, by the electronic device, image data including a face of the user;
identifying, by the electronic device, the face of the user; and
labelling, by the electronic device, the entry in the health datastore such that the event is identified with the user.

20. The one or more non-transitory computer-readable media of claim 16, wherein the action relating to the eye health event includes at least one of displaying a notification on the user device, occluding a portion of the display of the user device, turning off the display of the user device, activating one or more haptic devices included in the user device, or transmitting a notification to a computing device.
